(19) United States
(12) Patent Application Publication
(10) Pub. No.: US 2025/0090043 A1
(43) Pub. Date: Mar. 20, 2025

(54) System and Method of Synchronization of Location Signals

(71) Applicant: BIOSENSE WEBSTER (ISRAEL) LTD., Yokneam (IL)

(72) Inventors: Assaf Govari, Haifa (IL); Vadim Gliner, Haifa (IL); Andres Claudio Altmann, Haifa (IL)

(21) Appl. No.: 18/369,230

(22) Filed: Sep. 18, 2023

(51) Int. Cl. A61B 5/06; A61M 25/00

(52) U.S. Cl. CPC A61B 5/065; A61M 25/0082

(57) ABSTRACT

A methods and systems are disclosed to determine positions of one or more distal end portions of a catheter. In embodiments, the method includes processing location signals that are transmitted by a transmitter-receiver utility having a first clock, and received by a position sensor at a catheter's distal-end portion. The processing may be performed by a signal processor having a second clock not synchronized with the first clock of the transmitter-receiver utility. The processing includes: determining a clock-rate-ratio between clock-rates of the first and second clocks; determining a time-lag between the first and second clocks; and determining the relative phases of the received signals while compensating for the clock-rate-ratio and for the time-lag between the clocks, such that determined phases are adjusted relative to the first signal clock of the transmitter receiver utility. Accordingly, the position of the catheter's distal-end portion can be determined based on the relative phases.

### TECHNOLOGICAL FIELD

The present invention is in the field of medical devices' signals and particularly relates to determining the positions of multiple medical instruments or other portions thereof during medical procedures.

### BACKGROUND

A wide range of medical procedures involve a plurality of medical devices on and/or within a patient's body. The medical devices may be for example one or more catheters, and in many cases a single catheter includes a plurality of portions/sections, which may be for example one or more flexible sections (such as flexible arms/splines thereof) and/or medical instruments which may be distributed on the flexible sections (e.g., sensors/probs, electrodes such as EEG electrodes, ablation instruments and/or other instruments). During a medical operation the flexible sections of such catheters may be flexed/bent to simultaneously approach/contact different parts of the treated anatomy, for sensing (mapping) and/or treating the tissue at those different parts by the medical instruments thereon, in some case simultaneously. One medical procedure in which the use of catheters including a plurality of flexible sections/portions have proved extremely useful is in the treatment of cardiac arrhythmias. Cardiac arrhythmias and atrial fibrillation in particular, persist as common and dangerous medical ailments, especially in the aging population.

Diagnosis and treatment of cardiac arrhythmias include mapping the electrical properties of heart tissue, especially the endocardium and the heart volume, and selectively ablating cardiac tissue by application of energy. Often in such procedures, one or more catheters having a plurality of portions such as flexible sections/splines, are inserted into the patient body. In some cases, medical instruments arranged on these plurality of portions are operated for carrying out multiple interrelated operations such as electrocardiogram (ECG) mapping, tissue ablation, temperature sensing/mapping and/or image/ultrasound sensing. Some catheters are dedicated for placement in specific parts of the anatomy, e.g., heart chamber, coronary sinus, esophagus, atrium, ventricle, and include multiple flexible sections/portions that can conform to the shape of the anatomy of the treated body part, and optionally having a plurality instrument arranged at the different portions.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 is a schematic illustration showing a catheter-based mapping and ablation system according to an embodiment of the present invention;

FIG. 2 is a schematic illustration of a catheter having multiple portions/medical instruments at a distal end thereof and position sensors associated therewith;

FIG. 3A is a block diagram illustrating the configuration of a positioning system 100 according to an embodiment of the present invention;

FIG. 3B is a flow diagram illustrating a method 200 to determine positions of medical devices and/or of distal end portions thereof according to embodiments of the present invention;

In some figures, like reference numerals are used to designate similar modules/elements of the present invention or elements/modules having like functionalities. Accordingly, unless otherwise specified, description of modules/elements with reference to a certain embodiment of the invention should be understood to apply to all embodiments of the present invention in which such module/element is incorporated.

### DETAILED DESCRIPTION OF EMBODIMENTS

With advancements of medical technologies, the number of medical devices and medical instruments that are involved in a medical procedure and spatially arranged, placed or moved, within the patient's body has increased significantly. For example, in some medical procedures, catheters, such as intracardiac ECG mapping catheters and/or ablation catheters, having a plurality of distal end portions, such as movable/flexible sections (e.g., splines) and/or medical instruments are used. For example, in many cases the medical instruments are arranged at various movable/flexible sections of the catheter's distal tip, such as the shaft, distal end effector, splines and/or at various other sections of the distal end effector thereof. The movable/flexible sections of the catheter may be adapted for flexing/expanding during the medical procedure (e.g., in order to accommodate the volume of the treated/monitored body anatomy and/or for bringing medical instruments thereon into contact or close proximity, with the wall/boundary of the treated/monitored body anatomy. Such techniques are used for example in order to enable mapping and/or treating several tissue regions of the anatomy substantially simultaneously. To this end, a large number of medical instruments, such as ECG electrodes, ablation utilities/instruments or other instruments, which may be arranged at a plurality of movable/flexible sections of a catheter, and may for example be used to enable a physician to simultaneously capture/map the electrical activity from multiple locations in the heart chamber, and/or or to simultaneously ablate specific tissue regions thereof.

In order to achieve such medical procedures with accuracy, the positions of the medical instruments furnished at the different flexible/movable sections of the catheter, and/or the positions of one or more of the flexible/movable sections of the catheter themselves should be monitored/tracked relative to treated/probed anatomy of the patient's body.

For clarity, in the following, the phrase distal end portions of a medical device/catheter, and variants thereof is used to designate portions/elements/sections that are arranged at a distal tip/end of a medical device/catheter, and whose positions should be determined during a medical procedure. In this regard it should be understood that the phrase distal end portions may encompasses any one of flexible/movable section(s) of a catheter's distal tip, and/or medical instruments arranged thereon, which are associated with position sensors located adjacent thereto and capable of providing data/signals indicative of the respective positions of the distal end portions. The term position is used herein to designate any one of a location, orientation or both, of a medical device or of a distal end portion thereof. For instance, in some case there may not be a need to determine the location of a medical-device or of portion thereof, but only its orientation, or vice-versa, and the system of the present invention may be adapted/operable to achieve the same.

To achieve that medical device/catheter may be configured such that different distal end portions thereof, or some of them, include respective position sensors capable of receiving location signals (e.g., electromagnetic signals) by which the positions of the different portions can be determined. These received location signals should then processed in order to monitor/track the positions of different portions of the catheter's distal end a during the medical procedure. Accordingly in this way valuable information about the positions of different portions of the medical device/catheter can be provided to the physician during the medical procedure (e.g., in real-time).

In view of the above there is a need in the art for systems and methods to enable determining the positions of the plurality of portions of a distal end of a catheter or other medical devices having different portions thereof movable with respect to one another and/or with respect to a main body/shaft of the device. Conventional hardware for determining the positions of medical devices, are often limited in their capacity to determine a plurality of discrete positions from pluralities of position sensors such as those furnished on different distal end portions of catheters of the type described above (as various catheters of this type may include between few and few tens of position sensors). To this end, the conventional hardware of medical device position determination is generally not suited for connecting to medical devices such as catheters, having relatively large pluralities of position sensors whose positions need to be determined. Indeed, the conventional hardware typically includes transmitters for transmitting the location signals that can be received by the plurality position sensors of such catheters or other medical devices, but has limited channels for collecting the locations signals received by the plurality of position sensors of such devices, and cannot accommodate simultaneous processing of location signals provided from such pluralities of positions sensors that are embedded in some of the advanced medical devices, such as spline or basket type catheters which have a plurality of respectively movable portions at their distal end/tip.

The present invention therefore provides novel systems and methods for solving these problems to enable extending the capabilities of existing medical device positioning hardware to enable determining (e.g., simultaneously), the positions of multiple portions of medical devices, while utilizing, and in synchronization with, location signals that are provided/transmitted by the existing conventional medical device positioning hardware. Advantageously the invention facilitates the positioning of relatively large pluralities of position sensors which may not be facilitated by the conventional positioning hardware, while without a need to replace the existing positioning hardware (e.g., which may already be deployed) and without addition of location signal transmitters or transmission of additional location signal. Accordingly, the positions of large number of medical devices/instruments or portions thereof may be determined during a medical procedure in cost effective manner, and without overcrowding the medical facilities with additional electromagnetic signals.

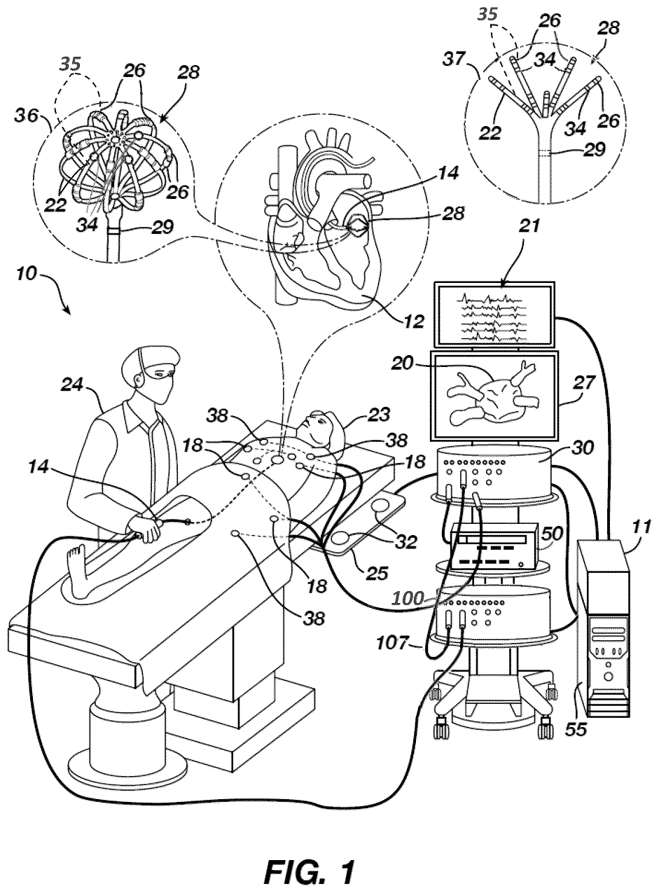

Reference is made to FIG. 1 showing an example catheter-based mapping and ablation system 10. System 10 includes or is connectable to multiple medical devices 14 and may include for example catheters 36 and 37, whose positions, and preferably the positions of the various distal end portions 35 thereof, should be determined by the system 10. It should be noted that the distal end portions 35 whose positions are to be determined, a may include the main body (e.g. a catheter's shaft) 14H of a medical device 14 such as catheter 36 or 37, and/or one or more flexible/movable sections/splines 22 thereof, and/or medical instruments 26 furnished at the distal end of the medical device 14 (e.g. being movable relative to the main body/shaft of the medical device 14) and whose positions therefore may not be accurately determined based only on measurements of the position of the medical device itself. In the none-limiting examples provided herein the distal end portions 35 include some of the medical instruments 26 and/or the movable sections/splines 22 at which they are furnished in the catheters 36 and/or 37. It should also be noted that the movable sections 22 may be implemented using various configurations such as flexible splines as shown in the figure, or by other configurations for example utilizing pivots, flexible joints, extendable/telescopic shafts or any other configuration without departing from the scope of the present invention.

Catheters 36 and/or 37 may be percutaneously inserted by a physician 24 through the patient's vascular system into a chamber or vascular structure of a heart 12. Typically, a delivery sheath catheter is inserted into the left or right atrium near a desired location in heart 12. Thereafter, one or more catheters, such as 36 and/or 37, may be inserted into the delivery sheath catheter so as to arrive at the desired location in heart 12. For example, the system 10 may include catheters dedicated for sensing Intracardiac Electrogram (IEGM) signals, such as catheter 37, catheters dedicated for ablating, such as catheter 36, catheters dedicated for both sensing and ablating (not specifically shown in the figure), and/or other medical devices. Examples for catheters 37 and 36, which are configured for sensing IEGM and for ablating are respectively illustrated herein. Physician 24 may place a distal tip 28 of any of the catheters 14 such that multiple medical instruments 26 thereof, which are arranged on the flexible/movable sections 22 of the catheter 14 can move/flex to approach/contact the heart wall for probing/treating target sites in heart 12. For sensing IEGM signals and/or for ablating a tissue, physician 24 may place a distal end 28 of the respective sensing or ablation catheter, 37 or 36, such that the medical instruments 26 thereof approach/contact with tissue at the target site.

Catheters 36 and 37 shown in the figure exemplify medical devices 14 whose positions, and/or the positions of at least some distal end portions 35 thereof (e.g., medical instruments 26 and/or movable sections 22) should be determined (tracked/monitored) by the system 10. Magnetic based position sensors 29 and/or 34 located on the catheter's distal tip 28 may be operated to provide data indicative of the real-time positions of the distal end portions 35 of the catheters during the medical procedure. The magnetic based position sensors 29 and 34 operate in conjunction with location pad 25 which includes a plurality of location signal transmitters 32 (e.g., magnetic coils) that generate/transmit electro-magnetic location signals (e.g., magnetic fields) in a predefined working volume surrounding the patient. Real time position of the distal tip 28 of the catheter 36/37 and/or of the distal end portions 35 thereof may then be tracked based on the electro-magnetic location signals that are generated with location pad 25 and sensed by magnetic based position sensor 29 and/or 34. Details of the magnetic based position sensing technology are described in U.S. Pat. Nos. 5,5391,199; 5,443,489; 5,558,091; 6,172,499; 6,239,724; 6,332,089; 6,484,118; 6,618,612; 6,690,963; 6,788,967; 6,892,091.

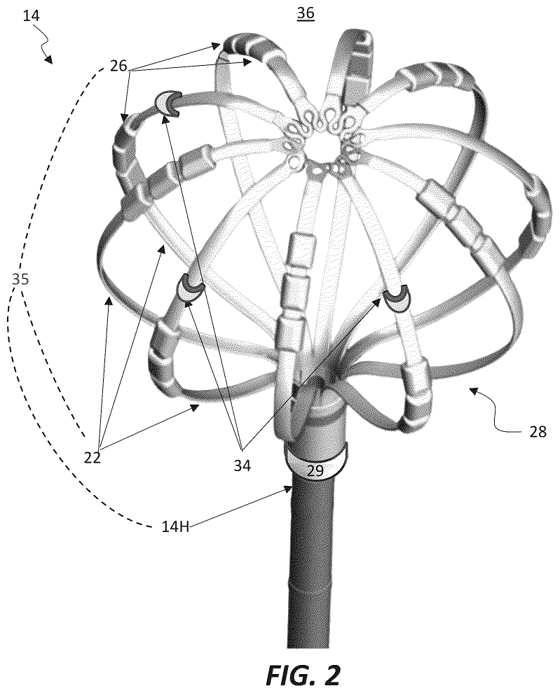

Catheters 36 and 37 shown in FIG. 1, exemplify medical devices 14 that are connectable to the system 10 and each includes multiple distal end portions 35 including in these examples flexible sections 22 (splines or basket arms) extending from the distal tips 28 of the catheters 36 and 37 and medical instruments 26 arranged thereon. With reference to FIG. 2, a perspective view of an ablation catheter 36 manifesting an example of a medical device 14 with movable distal end portions 35 is illustrated. The catheter 36 includes a shaft 14H having a distal end/tip 28. The catheter 36 in this non-limiting example includes a plurality of distal end portions 35 arranged at the distal end/tip 28 including flexible sections 22 (hereinafter also referred to as splines) and multiple medical instruments 26 arranged thereon. The splines 22 arranged near/at the distal end 28 form and expandable distal end assembly that can be deflectable outwardly from the catheter by moving the pusher rode 14P to thereby engage the medical instruments 26 to approach/contact the tissue wall of the heart for ablation of desired regions of the tissue. In some embodiments, such as in catheter 37, medical instruments 26 may include for example near-field intracardial electrogram sensors/electrodes for sensing near-field signals IEGM signals (e.g., activation biopotential signals) when the catheter is located intracardially and the instruments 26 are approached to the tissue. In the non-limiting example of FIG. 2, the flexible sections 22 are connected to one another in a “basket” like form. In other examples, the flexible sections 22 of the catheter may be connected to one another in a different configuration and/or may be independently movable such as in catheter 37 shown in FIG. 1, and/or may have different shapes/configurations (e.g., other than splines).

Thus, the system of the present invention may be configured and operable for determining the position of a medical device(s) 14 connected thereto; e.g., catheter(s) 36 and/or 37, and/or of distal end portions 35 thereof. The catheter, for example 36, may include a plurality of position sensors e.g., 29 and 34 enabling the system the system 10 of the present invention to determine the positions of distal end portions 35. In this none-limiting example catheter 36 includes a position sensor 29 embedded in the main body of the catheter (e.g., in or near the distal tip 28 thereof) to enable tracking the location and/or orientation of distal tip 28 of the catheter 36. Additionally, in this none-limiting example catheter 36 includes a plurality of position sensors 34 arranged at the movable sections 22 or other distal end portions 35 thereof and, whose positions can also be tracked by the system 10. The position sensors 29 and/or 34 are adapted to receive location signals that are transmitted by a location signal transmitter receiver utility of the system 10, and to provide the received signals for processing by system 10. The location signals obtained from the position sensors 29 and/or 34, enable the system 10 to determine the respective positions of some or all of the distal end portions 35 of the catheter 36, such as the positions of the flexible sections 22 thereof and/or the positions of medical instruments thereon 26, as well as typically the position of the main-body/distal-tip 28 of the catheter 36. In some embodiments the system 10 may utilize the signals from the position sensors 34 to determine/assess the shape(s) of the distal end flexible sections 22 of the catheter while they being flexed/shifted during the medical procedure.

Typically, the position sensors 29 and/or 34 are magnetic/electromagnetic based position sensors. In some embodiments, the position sensors such as 29 may be implemented as a three axes sensor (TAS) including three magnetic coils to enable sensing of three-dimensional (3D) location and orientation thereof. In some embodiments, the position sensors, such as 34, may be implemented as single-axis sensor (SAS) including one magnetic coil to enable axial sensing of their orientation (e.g., whereby the axial position sensing obtained by SAS sensors may be for instance combined with three-dimensional (3D) position and orientation sensing of another position sensor of the catheter, such as 29, to facilitate determining the locations of the SAS sensors). It should be noted that the catheter 37 illustrated in FIG. 1 may in some cases have similar configuration as catheter 36 describe above, although the flexible sections 22 may be configured differently, e.g., not necessarily connected/constrained to one another in basket form.

Turning back to FIG. 1, system 10 may also include one or more electrode patches 38 positioned for skin contact on patient 23 to establish location reference for location pad 25. Optionally in some embodiments one or more of the distal end portions 35 include ECG sensors/electrodes, and the location pad 25 may also be used for locating such distal end portions by impedance-based location tracking. Impedance-based location tracking may be used for example for locating distal end portions 35 that are not associated/furnished with position sensors capable of sensing their accurate positions, provided that the distal end portions 35 are furnished with electrodes suitable for determining their positions utilizing impedance-based tracking techniques. For impedance-based tracking, electrical current is directed to electrodes (e.g., ECG electrodes in case such are included at/on the distal end portion 35). The electrical current is then sensed at electrode skin patches 38 so that the location of each ECG electrode can be triangulated via the electrode patches 38. Details of the impedance-based location tracking technology are described in U.S. Pat. Nos. 7,536,218; 7,756,576; 7,848,787; 7,869,865; and 8,456,182.

System 10 may also include a recorder 11 records and displays electrograms 21 captured with body surface electrocardiogram (ECG) electrodes 18 and intracardiac electrograms (IEGM) captured with ECG electrodes of one of the medical devices/catheters 14 which may be connected to the system. Recorder 11 may include pacing capability for pacing the heart rhythm and/or may be electrically connected to a standalone pacer.

System 10 may include an ablation energy generator 50 that is adapted to conduct ablative energy to one or more of electrodes at a distal tip of one of the catheters 14 that configured for ablating. Energy produced by ablation energy generator 50 may include, but is not limited to, radiofrequency (RF) energy or pulsed-field ablation (PFA) energy, including monopolar or bipolar high-voltage DC pulses as may be used to effect irreversible electroporation (IRE), or combinations thereof.

System 10 typically include a Patient interface unit (PIU) 30, which is an interface configured to establish electrical communication between medical devices, such as catheters and/or other electrophysiological equipment, and a workstation 55 for controlling operation of system 10. The medical devices of system 10 include may include for example electrophysiological equipment such as one or more catheters 14, location pad 25, body surface ECG electrodes 18, electrode patches 38, ablation energy generator 50, and recorder 11. Optionally and preferably, PIU 30 additionally includes processing capability for performing ECG calculations and/or for implementing real-time computations of location of some of the medical devices/catheters connected to the system.

Workstation 55 includes memory, processor unit with memory or storage with appropriate operating software stored therein, and user interface capability. Workstation 55 may provide multiple functions, optionally including (1) modeling the endocardial anatomy in three-dimensions (3D) and rendering the model or anatomical map 20 for display on a display device 27, (2) displaying on display device 27 activation sequences (or other data) compiled from recorded electrograms 21 in representative visual indicia or imagery superimposed on the rendered anatomical map, (3) displaying real-time location and orientation of multiple catheters within the heart chamber, and (4) displaying on display device 27 sites of interest such as places where ablation energy has been applied. One commercial product embodying elements of the system 10 is available as the CARTO™ 3 System, available from Biosense Webster, Inc., 31A Technology Drive, Irvine, CA 92618.

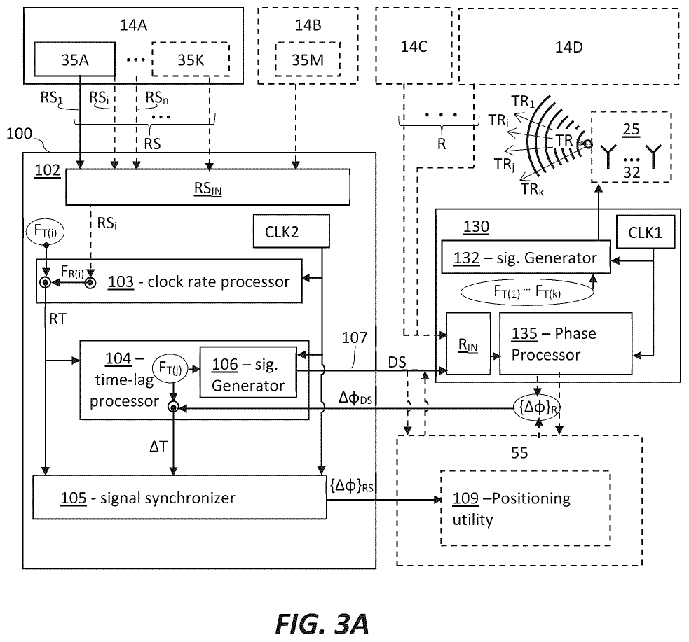

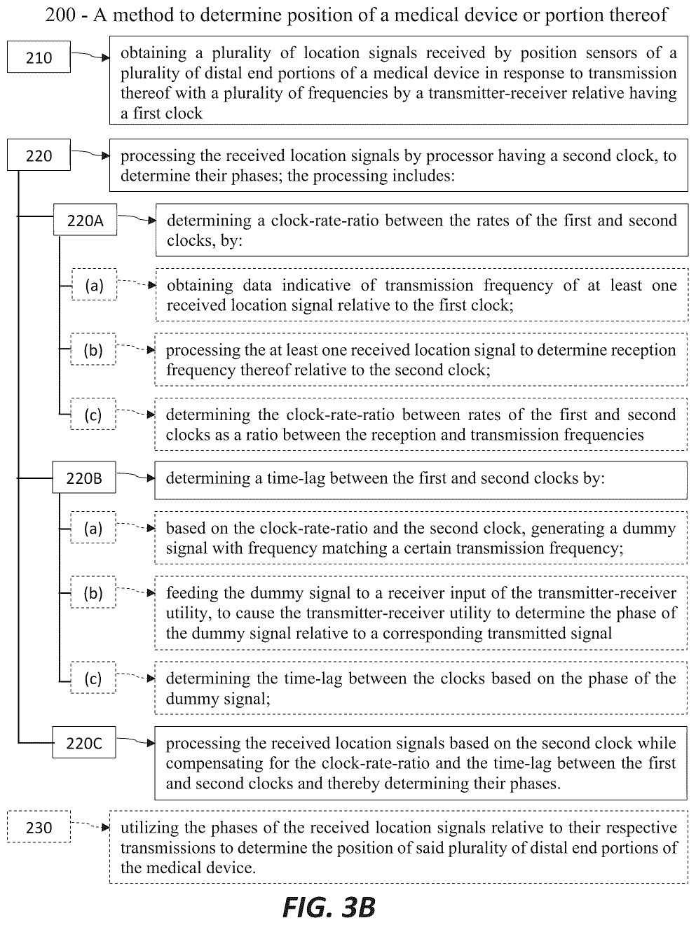

As indicated above, typically, the PIU 30 typically implements processing capability for implementing real-time computations of positions of some of the medical devices/catheters 14 that are connected to the system 10. To achieve that the PIU 30 typically includes a transmitter-receiver utility (not specifically shown in FIG. 1; 130 in FIG. 3A) capable of transmitting location signals and obtained the received signals which are received in responsive to said transmissions by positions sensors associated with the medical modules, and applying real-time processing to the received signals to determine data indicative of the positions of at least some of the medical devices 14, which are connected to the system 10. Specifically, the transmitter-receiver utility of the PIU 30 includes a signal generator that is connected to the location pad 25 and adapted to generate electromagnetic location signals of certain plurality of frequencies. The location signals are then transmitted by the location pad 25 and received by the position sensors 29 and/or 34 of the medical devices/modules 14/26 that are connected to the system 10.

In turn the transmitter-receiver utility of the PIU 30 is connected to some of the medical devices 14 and is adapted to collect and process the location signals that were received by their position sensors and determine phases relative to the transmitted location signals, based on which the positions of the medical modules/devices can be determined. The transmitter-receiver utility of the PIU 30 may be for example adapted to apply analog to digital conversion (sampling) to the position signals received from the position sensors of the medical devices that are connected thereto, to determine their phases relative to the location signal transmissions made thereby, and provide/communicate the same, (or the actual positions calculated from the phases) to the workstation 55, for further processing or display to the physician.

However, in some implementations of systems 10 (e.g., systems already deployed in medical operation facilities), the PIU 30 may have limited input channels for collecting and/or simultaneously sampling location signals and is not adapted to accommodate sufficient number of input channels for the multitude of position sensors which are included implemented in advanced medical devices 14 having a plurality of distal end portions 35 whose positions should be separately determined.

Therefore, in some implementation system 10 includes an additional positioning system 100 which is configured and operable for complementing/extending the functionality of the PIU 30 to enable connecting to the system 10, additional channels for position sensors of additional medical devices, such as catheters 36 or 37, which have one or more respectively movable distal end portions 35 at the distal end 28 thereof.

Reference is now made together to FIGS. 3A and 3B in which a block diagram of a positioning system 100 and a flow chart of a method 200 according to embodiments of the present invention are respectively illustrated. FIG. 3A depicts the configuration of the system 100, which is adapted to determine positions of a plurality of distal end portions 35A-35K and 35M of one or more medical devices e.g. 14A and 14B. FIG. 3B illustrated the method 200 for determining the positions of distal end portions such as 35A-35K and 35M. Method 200 is implemented by the system 100 and accordingly, for brevity embodiments of the system 100 and the method 200 of the present invention are described herein below together with reference to the FIGS. 3A and 3B.

The system 100 includes a signal processor 102 that is configured and operable for connection with the position sensors (e.g. 29 and/or 34) in FIG. 1 associated with one or more distal end portions, such 35A to 35K, 35M of medical devices 14A and/or 14B and/or other medical devices or distal end portions thereof, to receive therefrom a plurality of location signals RS (hereinafter also refer to as received signals) received by position sensors 29/34 associated therewith, and determine the phases of the received location signals RS, by which the positions of the distal end portions or some of them can be inferred.

In the present non-limiting example, medical modules 35A to 35K, 35M are shown to be associated-with/parts of medical devices 14A and 14B respectively. The position sensors of the medical devices 14A and 14B and/or of the distal end portions thereof, 35A to 35K and 35M, are connected to signal processor 102 for inference of data indicative of their respective positions, by the signal processor 102. It should be understood that the present invention may be implemented with a different number of medical devices and associated distal end portions 35 that can be connected to the signal processor 102, and the specific number of medical devices and distal end portions shown in the figure are provided herein as an example and should not be limiting.

In FIG. 3A, additional medical devices 14C and 14D which may each have one or more position sensors at the distal tip thereof are illustrated as connected to a location transmitter receiver utility 130 (e.g., of PIU 30 shown in FIG. 1). Typically, the location transmitter-receiver utility 130, which may or may not be a part of the system 100, includes a signal generator 132 that is connectable to a plurality of location signal transmitters 32, which are typically arranged in location pad 25 (e.g., the location pad 25 is intended for placement near/underneath a patient being treated to enable locating the medical devices used in the medical procedure). The signal generator 132 of the transmitter-receiver utility 130 is configured and operable to generate a plurality of location transmission signals TR₁ to TRₖ (hereinafter transmissions or transmitted signals) having a plurality of respective transmission frequencies F_{T(1)} to F_{T(k)} (e.g., being predetermined frequencies). The signal generator 132 provides the transmission signals TR₁ to TRₖ to the location signal transmitters 32, by which they are transmitted as wireless electromagnetic signals (e.g., magnetic fields). In some embodiments the transmission signals TR₁ to TRₖ are continuous-wave (CW) signals having the different frequencies F_{T(1)} to F_{T(k)}.

In turn, one or more of the transmitted signals TR₁ to TRₖ are received/sensed by the position sensors of the medical devices 14A to 14D whose positions of the positions of one or more distal end portions thereof are to be determined. The transmitter-receiver utility 130 typically includes receiver input ports/channels R_{IN} and a phase processor 135 by which it can be connected to one or more (typically a plurality) of the medical devices, such as to medical devices 14C and 14D (i.e., to the position sensors associated therewith), to obtain the locations signals R received thereby. The received location signals R from medical devices 14C and 14D are processed by the transmitter-receiver utility 130 to determine their respective phases {Δϕ}_{R} relative to the corresponding transmitted signals TR₁ to TRₖ. The processing in this case, is made by transmitter-receiver utility 130 and therefore relies on the same clock CLK1 of the transmitter-receiver utility 130 which is used for the generation of the transmitted signals TR₁ to TRₖ. Accordingly, determining the relative phases of the location signals R received by the position sensors of the medical devices 14C and 14D that are connected transmitter-receiver utility 130 can be done accurately in straight forward manner by using the same clock which is used for the signals' transmissions. As would be readily appreciated by those versed in the art, in this manner the positions of these medical devices 14C and 14D can be inferred based on the relative phases of the received location signals R that are determined by the transmitter-receiver utility 130.

However, as indicated above, the transmitter-receiver utility 130 has a limited number of input ports/channels R_{IN} and can obtain and process only a limited number of received location signals R from a limited number of position sensors of the medical devices such as 14C and 14D. With the advent of medical technologies and the increase in numbers of position sensors such as 29 and 34 furnished at pluralities of distal end portions of medical devices, such as 14A and 14B, there is a need to supplement the signal processing capacity of the transmitter-receiver utility 130 to facilitate inference of the positions of the pluralities of distal end portions 35A-35K and 35M of such devices (since their number is often in excess of the number of input channels R_{IN} of the transmitter-receiver utility 130).

In order to achieve that, the system 100 includes the signal processor 102 indicated above. The signal processor 102 has additional input channels/ports RS_{IN} for receiving additional location signals RS from the positions sensors 29/34 that are associated with the distal end portions e.g., 35A-35K and 35M of one or more of the medical devices 14A and/or 14B that are connected to the system 100. In this regard it should be noted that hereinafter the phrases received signals and/or location signals designated by RS are used to refer to the location signals received from the position sensors furnished at medical devices such as 14A and/or 14B and/or on the distal end portions thereof 35A to 35K and 35M, which are connected to the signal processor 102 (in other words the location signals RS should be distinguished from the location signals R of the medical devices such as 14C and 14D which are processed by the transmitter-receiver utility 130). Having said that, it should also be noted that the transmitted signals TR₁ to TRₖ that are used for the positioning of the medical devices 14A and 14B (e.g. and/or the distal portions 35A to 35K and 35M thereof) whose position sensors are connected to the signal processor 102, are typically the same signals TR₁ to TRₖ that are transmitted by transmitter-receiver utility 130 and used for the positioning of the medical devices 14C and 14D whose positions sensors are connected to the transmitter-receiver utility 130. This is preferable in various implementations of the system for example for reasons of cost effectiveness as well as in order not to over crowd the medical operation facility with additional electromagnetic signals and additional equipment for their generation.

The signal processor 102 is thus configured and operable to process the location signals RS received/sensed by the position sensors of the medical devices 14A and 14B to infer data indicative of their positions and/or of the positions of the distal end portions thereof. To this end, in line with operations 210 of method 200, the signal processor 102 obtains the received location signals RS from the position sensors associated with distal end portions 35A-35K and 35M of the medical devices 14A and 14B connected thereto, for example via the input channels/ports RS_{IN} of the signal processor 102. The signal processor 102 then implements the operation 220 of method 200 and processes the location signals RS to determine their respective phases {ϕ}_{RS} relative to corresponding ones of the transmitted signals TR₁ to TRₖ which have the same frequencies.

As indicated above, the frequencies of the transmitted signals TR are typically set by the transmitter-receiver utility 130 based on the first clock CLK1 being the clock of the transmitter-receiver utility 130, while the processing of the received location signals RS is in this case performed by the signal processor 102 based on a second clock CLK2 being the clock of the signal processor 102. The first and second clocks, CLK1 and CLK2, are not necessarily synchronized and may generally operate with somewhat different respective first and second clock rates and/or have time-lag between them. Therefore, a naïve attempt to infer the frequencies of the received signals RS based on the clock CLK2 of the signal processor 102 (and thereby also infer their phases), would generally result with a discrepancy between the frequencies of the transmitted signals TR and the inferred frequencies of the corresponding received signals RS. This may in turn yield erroneous inferred relative phases between them (e.g., as inference of the phase depends on frequency as well as the time-lag/difference between the clocks).

The signal processor 102 is therefore configured and operable mitigate such discrepancies of frequencies and/or time lag between the clocks, which might be caused by the lack of clock synchronization. In some embodiments the signal processor 102 is configured and operable as follows according to operation 220 of method 200 in order to determine the phases {ϕ_{RS}} of the received signals RS relative to their respective transmissions TR₁ to TRₖ, with accuracy. The signal processor 102 includes: a clock rate processor 103 that is adapted to implement the operation 220A of method 200 and determine the clock-rate-ratio RT between the first rate of the first clock CLK1 of the transmitter-receiver utility 130 and the second rate of the second clock CLK2 of the signal processor 102; a time lag processor 104 that is adapted to implement the operation 220B of method 200 to determine the time-lag ΔT between the first and second signal clocks, CLK1 and CLK2, of the transmitter-receiver utility 130 and the signal processor 102 respectively; and a signal synchronizer 105 that is adapted to implement the operation 220C of method 200 and digitize the received signals RS with compensation for the clock-rate-ratio RT and the time-lag ΔT determined by the clock rate processor 103 and the time lag processor 104 respectively.

More specifically, in some embodiments the clock rate processor 103 is configured and operable to determine the clock-rate-ratio RT between the first and second clock-rates, by carrying out the following:
(a) obtain data indicative of a transmission frequency F_{T}(i) of at least one received location signal e.g., R_{Si} of the location signals RS, relative to the first clock-rate of the first clock CLK1 of the transmitter-receiver utility 130 (i.e., obtain data indicative of the frequency F_{T(i)} by which the transmitted signal TRᵢ which corresponds to the at least one received signal R_{Si} was transmitted by the transmitter-receiver utility 130). This generally implements the operation 220A-(a) of method 200.
(b) process the at least one received location signal RS; to determine a reception frequency F_{R(i)} thereof relative to the second clock-rate of the second clock CLK2 of the signal processor 102—thus generally implementing the operation 220A-(b) of method 200; and
(c) determine the clock-rate-ratio RT based on the transmission and reception frequencies, F_{T(i)} and F_{R(i)}, that are inferred for the at least one location signal RS; in (a) and (b) respectively. This generally implements the operation 220A-(c) of method 200.

The operation 220A and its optional sub operations implemented by the clock rate processor 103 may be carried out for example based on the following. As for the transmission frequency F_{T(i)} obtained in (a), in typical embodiments where the transmitter-receiver utility 130 utilizes predetermined/preset transmission frequencies F_{T(1)} to F_{T(k)} for the transmitted signals, the clock rate processor 103 may be preconfigured or adjusted to operate according to a certain one F_{T(i)} (or more) of these predetermined/preset transmission frequencies. Alternatively, in cases where the transmission frequencies F_{T(l)} to F_{T(k)} are not a-priori fixed, the clock rate processor 103 may be adapted to obtain data indicative of one or more of them from an external utility such as the transmitter-receiver utility 130 itself or from another utility, such as workstation 55, which may hold information about the transmission frequencies by which the transmitter-receiver utility 130 operates.

As for the reception frequency F_{R(i)}, inferred in (b), different techniques for inferring this frequency F_{R(i)} from the received signal RSᵢ may be implemented in various embodiments of the invention. For instance, in some embodiments the clock rate processor 103 digitizes the received signal RSᵢ (e.g., samples it) based on the clock rate of the second clock CLK2 and thereby yields a digitized received signal. Then the clock rate processor 103 applies tone detection processing (e.g., spectral analysis), such as Fourier-Transform (e.g., FFT), to the digitized signal, and thereby determines the frequency F_{R(i)} of the received signal RSᵢ relative to the second clock CLK2. As would be appreciated by those versed in the art, in this manner the frequency of the received signal RSᵢ relative to the second clock can be determined by identification of a prominent peak in the Fourier-Transform. Alternatively, or additionally, in some embodiments the clock rate processor 103 include a Clock-Derivation processor (not specifically illustrated in the figure; typically comprising a comparator and a counter) that is connected to the second clock CLK2 and to the input channels/ports RS_{IN} by which the received signal RSᵢ is obtained. Typically, the Clock-Derivation processor is adapted to determine a tick-count of the second clock CLK2 between zero-crossings of the received signal RSᵢ and thereby infer the frequency F_{R(i)} of the received signal RSᵢ relative to second clock CLK2 rate (in such implementations where the frequency is determined by Clock-Derivation, digitization and/or Fourier transform of the signal which may be computationally intensive, may be obviated).

Finally, the clock-rate-ratio RT between the first clock of transmitter-receiver utility 130 and the second clock CLK2 of the signal processor 102 may be for example determined in (c) according to the ratio between the frequency F_{R(i)} of the received signal RSᵢ as determined in (b) and the corresponding transmission frequency F_{T(i)} determined in (a). As both these frequencies pertain to the same underlying signal, but measured by the different respective second and first clocks, CLK2 and CLK1, their ratio is indicative of the ratio RT between the clock respective rates.

After the clock-rate-ratio RT is determined by the clock rate processor 103, the operation 220B and optionally its sub operations, is implemented by the clock rate processor time lag processor 104 as described in the following. The time lag processor 104 operates to determine the time-lag ΔT between the clock CLK1 of the transmitter-receiver utility 130 and the clock CLK2 of the signal processor 102. To achieve that in some embodiments the time-lag processor 104 is configured and operable to carry out the following optional operations:
(a) Obtains the clock-rate-ratio RT determined by the clock rate processor 103, and based on the clock-rate-ratio RT, generates a dummy signal DS with frequency that matches a certain one of the transmission frequencies of the signals TR₁ to TRₖ transmitted by the transmitter-receiver utility 130. This generally implements the operation 220B-(a) of method 200. In this non-limiting example, the dummy signal DS is generated with frequency F_{T(j)} that matches the frequency of the transmitted signal TRⱼ.
(b) The time lag processor 104, then feeds the dummy signal DS to the receiver input Rin of the transmitter-receiver utility 130. As indicated above this generally causes the transmitter-receiver utility 130 to determine the phase difference Δϕ_{DS} between the dummy signal DS and the corresponding transmitted signal TRⱼ that is transmitted thereby with the same frequency F_{T(j)}. This generally implements the operation 220B-(b) of method 200.
(c) Subsequent to the feeding of the dummy signal DS, the time lag processor 104 implements operation 220B-(c) of method 200. The time lag processor 104 obtains, directly or indirectly (e.g. via workstation 55), the phase difference Δϕ_{DS} that is determined by the transmitter-receiver utility 130 between the dummy signal DS and the corresponding transmitted signal TRⱼ, and utilizes the frequency value F_{T(j)} of dummy signal DS and the phase difference Δϕ_{DS} determined by transmitter-receiver utility 130 to infer the time-lag ΔT between the clock CLK1 of the transmitter-receiver utility 130 and the second clock CLK2 of the signal processor 102. The time-lag ΔT between the clocks CLK1 and CLK2 may for example be computed as:

ΔT=Δϕ_{DS}/2πF_{T(j)}.

More specifically in some embodiments the operation of the time lag processor 104 and operation 220B of method 200 may be carried out for example as follows. The time lag processor 104 may include a signal generator 106 that is connected to the clock CLK2 of the signal processor 102 (i.e., to the second clock). In 220B-(a) the time lag processor 104 may operate the signal generator 106 to generate the dummy signal DS with the desired transmission frequency F_{T(j)}. In this regard it should be understood that as the frequency of the dummy signal should match one of the transmission frequencies of the transmitter receiver utility 130, e.g., match the frequency F_{T(j)}. Therefore, as this frequency F_{T(j)} is set relative to the clock CLK1 of the transmitter receiver utility 130, in order to generate the dummy signal DS with that frequency based on the signal processor's second clock CLK2, the time lag processor 104 implements a clock-rate compensation to compensate for the difference between the clock-rate of the second clock CLK2 that is used for the dummy signal's DS generation, and that of the first clock of CLK1 of the transmitter receiver utility 130. The clock-rate compensation is based on the clock-rate-ratio RT between the first and second clocks CLK1 and CLK2 as determined by the clock rate processor 103, and in various embodiments of the present invention may be implemented for example by any of the following techniques or by combination thereof. (i) by adjusting the rate of the second clock CLK2 by a factor of said clock rate ratio RS such that it matches the rate of the first clock CLK1; and/or or (ii) by utilizing the second clock CLK2 (e.g. without adjusting its rate) but applying the compensation by generating the dummy signal DS with frequency F_{T(j)} multiplied by the clock rate ratio RS. Accordingly, the dummy signal is generated with the frequency Fro) set relative to the first clock CLK1 of the transmitter receiver utility 130 such that when it is processed by the transmitter receiver utility 130 based on the first clock CLK1, the transmitter receiver utility 130 would “identify” it as having frequency F_{T(j)} matching one of its transmitted signals and would determine its relative phase accordingly.

In some implementations the system 100 includes a signal channel 107 (e.g., signal cable/communication-path) of substantially fixed and predetermined latency Δt, adapted for interconnecting the time lag processor 104 directly or indirectly (e.g., via workstation 55) to an input channel R_{IN} of the transmitter receiver utility 130. In such embodiments the time lag processor 104 is adapted to feed the dummy signal DS as input to the transmitter receiver utility 130 via that signal channel 107 having the substantially fixed latency. Then, upon receiving and processing the phase difference Δϕ_{DS} of the dummy signal DS to determine the time-lag ΔT, the time lag processor 104 accounts for the fixed predetermined latency Δt of the signal channel 107 (e.g., and subtracts the latency from the computed time-lag ΔT to thereby obtain ΔT more accurately as: ΔT=Δϕ_{DS}/2πF_{T(j)}−Δt.

Subsequent to the operations of the clock-rate and time-lag processors, 103 and 104, the signal synchronizer 105 implements the operation 220C of method 200 in order to determine the phases Δϕ_{RS} of the received location signals RS relative to their respective transmissions TR₁ to TRₖ by the transmitter receiver utility 130 so that the position(s) of the medical modules that are connected to the signal processor 102 can be inferred based on these phase Δϕ_{RS}. The signal synchronizer 105 processes the received location signals RS based on the second clock CLK2, the clock rate-ratio RT that was determined by the clock rate processor 103 and the time-lag ΔT that was determined by the time lag processor 104, to determine the phases Δϕ_{RS} of the received signals RS.

For instance, in some embodiments the signal synchronizer 105 digitizes the received location signals RS utilizing the second clock CLK2 and utilizes the clock rate-ratio RT and the time-lag ΔT to compensate for the difference in clock rates RT and for the time-lag ΔT between the clocks CLK1 and CLK2. Accordingly in such embodiments, the signal synchronizer 105 yields respective digitized received signals whose frequencies and phases adjusted relative to the first signal clock CLK1 of the transmitter receiver utility 130. Then, once the frequencies and phases of the received signals (or digitized versions thereof) are adjusted relative to the first signal clock CLK1, the signal processor 102, or the signal synchronizer 105 may process the signals (e.g., utilizing tone detection (e.g., FFT) or clock derivation, as described above, to determine their phases {Δϕ}_{RS} relative to the respective transmission signals that are transmitted by the transmitter receiver utility 130.

To achieve that, in some implementations the signal synchronizer 105 is adapted to compensate for the difference in clock rates by adjusting the rate of the second clock CLK2 in order to reduce or eliminate the difference between its rate (the second rate) and the rate of the first clock CLK1 of the transmitter receiver utility 130. The second clock's CLK2 rate may for example be adjusted by a factor of the clock rate ratio RS such that it matches the rate of the first clock CLK1. Alternatively, or additionally, the signal synchronizer 105 may be adapted digitize the received signals RS based on the second clock CLK2 (e.g. without rate adjustment of the clock) and then compensate for the difference RT in clock rates by interpolating the digitized signals based on the clock-rate-ratio RS (e.g. the “interpolation” here may in some cases be implemented by mere adjustment of the time labels of the signal samples by a factor of said clock-rate-ratio RS, which is a light weight computational process, or by interpolation of the samples themselves). Accordingly, in any of these exemplified techniques, the received signals RS are obtained as interpolated digital signals with their sample intervals (and hence frequencies) corresponding to the first clock rate of the first clock of the transmitter receiver utility 130.

Moreover, the signal synchronizer 105 may be for example adapted to compensate for the time-lag ΔT between the first and second clocks, CLK1 and CLK2, by adjusting the phase/timing of the second clock CLK2 in order to reduce or eliminate the time-lag ΔT. Alternatively, or additionally, the compensation for the time lag ΔT may be carried out by interpolating the digitized signals to shift their samples (or sample labels) according to the time-lag ΔT and thereby yield the digitized signals as interpolated digital signals with samples' times shifted (and hence phases adjusted) relative to the timing of the first clock CLK1 of the transmitter receiver utility 130).

To this end, the phases {Aϕ}_{RS} of the received location signals RS that are determined by the signal processor 102 according to the above technique relative to their respective transmissions by the transmitter receiver utility 130 are suitable for further processing to determine the positions of the position sensors 29 and/or 34, of the medical devices 14A and 14B and their associated distal end portions 35A to 35K and 35M, which are connected to the signal processor 102.

In some embodiments the system 100 further implements the optional operation 230 of method 200, and optionally includes a positioning utility 109 that is configured and operable to determining the positions of the positions sensors 29 and/or 34 of the medical devices 14A and 14B and their respective distal end portions 35A to 35K and 35M based on the phases {Aϕ}_{RS} inferred for their received signals RS by the signal processor 102. The positioning utility 109 may be part of the signal processor 102, or a part of a workstation 55 connected to signal processor 102 for receiving the phases {Aϕ}_{RS}. In some embodiments the positioning utility 109 may be adapted to also process the phases {Aϕ}_{R} of the received signals R that are received/sensed by position sensors of the medical devices 14C and/or 14D which are inferred, as indicated above, by the transmitter-receiver utility 130. To this end the positioning utility 109 may serve for determining the positions of all the position sensors 29 and/or 34 of the medical devices, which are connected to the system, irrespectively of whether the relative phases of the location signals R/RS sensed thereby are determined by the transmitter-receiver utility 130 or by the signal processor 102 of the present invention.

As will be readily appreciated by those versed in the art the position determination itself relative the respective positions of the signal transmitters, e.g. 32, may be implemented by various known in the art techniques (as long as the phases are correctly measured relative to the corresponding transmission signals), for example utilizing time-of-arrival (TOA), difference-time-of-arrival (DTOA), triangulation, and or any other suitable techniques or combinations thereof to determine the location(s) and/or the orientation(s) the medical module(s) relative to a reference frame (coordinates) defined by the signal transmitters e.g. 32. The positioning utility 109 is therefore configured and operable accordingly to determine the positions of the position sensors 29 and/or 34 of with the medical devices connected to the system 10. Accordingly, the positions of the medical devices 14A and 14B and/or of distal end portions thereof e.g., 35A-35K and/or 35M, can be determined by the system 100.

In this regard, turning back to FIGS. 1 and 2, as indicated above, in some implementations, a medical device 14 may be configured with a position sensor 29 arranged on a main body thereof (e.g., shaft of the catheter), while one or more additional position sensors 34 may be furnished on other distal end portions 35 of the medical device 14, such as on flexible sections 22 of the medical device, and/or on medical instruments 26 located thereon. In some implementations one or more of the position sensors 29 and/or 34 may be implemented as single-axis sensors (SAS) and may be utilized by the system to determine the orientation of the distal end portion at which they are furnished. In some implementations one or more of the position sensors 29 and/or 34 may be implemented as three-axes sensors (TAS) which can be used for measuring both the location and orientation of the distal end portion at which they are furnished. Accordingly, as indicated above the phrase position as used herein should be understood to refer to any of a location, orientation or both of a distal end portion 35 of a medical device 14.

To this end, it should be noted that in some implementations a medical device connected to the system may include at least one position sensor three-axes sensors (TAS) such as 29 and one or more single-axis position sensors (SAS) such as 34, arranged at distal end portions thereof. In this case the locations of one or more of the single-axis position sensors (SAS) 34 on the distal end portions may be determined by the positioning utility based on the inferred location and orientation of the three-axes sensors (TAS) e.g., 29, the inferred orientations of the one or more of the single-axis position sensors (SAS) 34, and a priory data indicative of the configurations/shape that the medical device may acquire/conform to during the medical procedure.

### EXAMPLES

Example 1. A method 200 to determine positions of one or more portions 35 of a catheter's 14 distal end 28, the method includes:
by a position sensor 29 and/or 34 at least a portion 35 of the catheter's distal end 28, receiving a plurality of location signals RS being wireless electromagnetic signals TR₁ to TRₖ that were transmitted by a transmitter-receiver utility 130 with respective plurality of transmission frequencies F_{T(1)} to F_{T(k)} relative to a first clock-rate of a first clock CLK1 associated with the transmitter-receiver utility 130; and
processing (220) the location signals RS by a signal processor 102 associated with a second signal clock CLK2 not synchronized with the first clock CLK1 and having a second clock-rate, to determine phases {Aϕ}_{RS} of the location signals RS relative to their respective transmissions TR₁ to TRₖ by the transmitter-receiver utility 130, the processing includes:
   A. determining (220A) a clock-rate-ratio RT as a ratio between the rates of the first and second clocks, CLK1 and CLK2;
   B. determining (220B) a time-lag ΔT between the first and second signal clocks, CLK1 and CLK2; and
   C. digitizing the location signals RS based on the second clock CLK2 to yield respective digitized signals. The digitizing includes (220C) compensating for the clock-rate-ratio RT and compensating for the time-lag ΔT, such that frequencies and phases of the digitized signals are adjusted relative to the first signal clock CLK1 of the transmitter receiver utility 130.

Accordingly determining the phases {Aϕ}_{RS} of the location signals RS relative to their respective transmissions TR₁ to TRₖ by the transmitter receiver utility 130 and enabling to determine the position of the at least a portion 35 of a catheter's 14 distal end 28 based on the determined phases {Aϕ}_{RS}.

Example 2. The method according to example 1, wherein the determining of the clock-rate-ratio between the first and second clock-rates, includes the following:
(a) providing data indicative of transmission frequency of at least one location signal of the location signals relative to the first clock-rate of the transmitter-receiver utility (based on which it is transmitted);
(b) processing the at least one location signal by the signal processor and determining the reception frequency thereof relative to the second clock-rate; and
(c) determining the clock-rate-ratio based on the transmission and reception frequencies.

Example 3. The method according to example 2, wherein the determining of the reception frequency of the at least one location signal relative to the second clock-rate includes digitizing the location signal based on the second clock to yield a digitized signal and applying tone detection processing, such as FFT, to the digitized signal.

Example 4. The method according to example 2 or 3, wherein the determining of the reception frequency of the at least one location signal relative to the second clock-rate includes applying Clock-Derivation processing to the at least one location signal.

Example 5. The method according to example 4, wherein the Clock-Derivation processing includes determining a tick-count of the second signal clock between zero-crossings of the at least one location signal and thereby determining the reception frequency thereof relative to the second signal clock.

Example 6. The method according to any of examples 1 to 5, wherein the determining of the time-lag between the first and second signal clocks, includes the following:
(a) utilizing the clock-rate-ratio for generating, by the signal processor, a dummy signal with frequency that matches a certain transmission frequency of the transmission frequencies of the location signals, and feeding the dummy signal to a receiver input of the transmitter-receiver utility, to thereby cause the transmitter-receiver utility to determine a phase difference between the dummy signal and one of the wireless electromagnetic signals that is transmitted thereby with that certain transmission frequency; and
(b) determining the time-lag between the clocks based on the phase difference and the certain transmission frequency.

Example 7. The method according to example 6, wherein the feeding of the dummy signal is carried out utilizing a channel of substantially fixed latency, and wherein the fixed latency is accounted for in the determining of the time-lag.

Example 8. The method according to example 6 or 7, wherein the generating of the dummy signal includes compensating for the clock-rate-ratio to thereby obtain the dummy signal with frequency matching the certain transmission frequency.

Example 9. The method according to any one of examples 6 to 8, including providing the phase difference between the dummy signal and the one of the wireless electromagnetic signals to the signal processor for the determining of the time-lag.

Example 10. The method according to any one of examples 1 to 9, wherein the compensating for the clock-rate-ratio includes adjusting the second clock-rate of the second signal clock in order to reduce or eliminate a difference between the first and second clock rates.

Example 11. The method according to any one of examples 1 to 10, wherein the compensating for the time lag includes adjusting the phase of the second signal clock in order to reduce or eliminate the time lag.

Example 12. The method according to any one of examples 1 to 11, wherein the compensating for the clock-rate-ratio includes re-interpolating the digitized signals based on the clock-rate-ratio to yield the digitized signals with samples corresponding to a sampling rate of the first signal clock.

Example 13. The method according to any one of examples 1 to 12, wherein the compensating for the time lag includes re-interpolating the digitized signals to shift their phases according to the time lag and thereby yield the digitized signals with phases adjusted relative to their respective transmissions by the transmitter receiver utility.

Example 14. The method according to any one of examples 1 to 13, further include determining the position of the at least portion of the catheter's distal end based on the phases of the received location signals, whereby the position is indicative of at least one of a location and orientation of the at least portion of the catheter's distal end relative to one or more reference-frame coordinates.

Example 15. A system 100 to determine a position of one or more portions 35 of a catheter's 14 distal end 28, the system includes:
a signal processor 102 configured and operable for connecting to a position sensor 29 and/or 34 arranged at an at least a portion 35 of a catheter's 14 distal end 28, for receiving, from the position sensor 29 and/or 34, a plurality of location signals RS indicative of electromagnetic signals TR₁ to TRₖ transmitted by a transmitter-receiver utility 130 with respective plurality of transmission frequencies F_{T(1)} to F_{T(k)} relative to a first clock-rate of a first clock CLK1 of the transmitter-receiver utility 130, and received by the position sensor 29 and/or 34;
The signal processor 102 includes a second signal clock CLK2 having a second clock-rate not synchronized with the first clock CLK1. The signal processor 102 is configured and operable to process the location signals RS to determine their phases {Aϕ}_{RS} relative to their respective transmissions TR₁ to TRₖ by the transmitter-receiver utility 130. The signal processor 102 includes:
   A. a clock rate processor 103 adapted to determine a clock-rate-ratio RT between the clock-rates of the first and second clocks, CLK1 and CLK2;
   B. a time lag processor 104 adapted to determine a time-lag ΔT between the first and second clocks, CLK1 and CLK2; and
   C. a signal synchronizer 105 configured and operable for utilizing the second clock CLK2, the clock-rate-ratio RT and the time-lag ΔT, to digitize the location signals RS with compensation for the clock-rate-ratio RT and with compensation for the time-lag ΔT, to thereby yield respective digitized signals with frequencies and phases adjusted relative to the first signal clock CLK1 of the transmitter receiver utility 130.

The signal processor thereby determines the phases {Aϕ}_{RS} of the location signals RS relative to their respective transmissions TR₁ to TRₖ by the transmitter receiver utility 130 and enables to determine the position of the at least portion 35 of the catheter's 14 distal end 28 based on the determined phases {Aϕ}_{RS}.

Example 16. The system according to example 15, wherein the clock rate processor is configured and operable to determine the clock-rate-ratio by carrying out the following:
(a) obtaining data indicative of a transmission frequency of at least one location signal of the location signals relative to the first clock-rate of the transmitter-receiver utility;
(b) processing the at least one location signal to determine a reception frequency thereof relative to the second clock-rate; and
(c) determining the clock-rate-ratio based on the transmission and reception frequencies of the at least one location signal.

Example 17. The system according to example 16, wherein the clock rate processor is adapted to determine the reception frequency of the at least one location signal relative to the second clock-rate by digitizing the location signal based on the clock rate of the second clock to yield a digitized signal, and apply tone detection processing to the digitized signal to thereby determine the frequency of the location signal.

Example 18. The system according to example 16 or 17, wherein the clock rate processor includes a Clock-Derivation processor that is adapted to determine the reception frequency of the at least one location signal relative to the second clock-rate. The Clock-Derivation processor is configured and operable to determine a tick-count of the second signal clock between zero-crossings of the at least one location signal and to thereby determine the receipt frequency of the at least one location signal relative to the second signal clock.

Example 19. The system according to any one of examples 15 to 18, wherein the time lag processor is configured and operable to determine the time-lag by carrying out the following:
(a) obtaining the clock-rate-ratio from the clock rate processor and utilizing the clock-rate-ratio to generate a dummy signal with frequency matching a certain transmission frequency of the transmission frequencies;
(b) feeding the dummy signal to a receiver input of the transmitter-receiver utility, to thereby cause the transmitter-receiver utility to determine a phase difference between the dummy signal and one of the wireless electromagnetic signals which is transmitted thereby with that certain transmission frequency; and
(c) obtaining that phase difference and determining the time-lag based on that phase difference and the certain transmission frequency.

Example 20. The system according to example 19, may include a signal channel of substantially fixed and predetermined latency interconnected directly or indirectly between the signal processor and the transmitter receiver utility. The time lag processor may be adapted to feed the dummy CW signal to the transmitter receiver utility via the channel of the substantially fixed latency, and to account for the fixed latency of the channel in determination of the time-lag.

Example 21. The system according to example 19 or 20, wherein the time lag processor is adapted to carry out the compensation for the clock-rate-ratio when generating the dummy signal, such that the dummy signal is generated with frequency that matches the certain transmission frequency.

Example 22. The system according to any one of examples 19 to 21, includes a direct or indirect connection with the transmitter receiver utility. The time lag processor is adapted utilize the connection to obtain the phase difference between the dummy signal and the one of the wireless electromagnetic signals that is transmitted by the transmitter receiver utility with similar frequency as that of the dummy signal, and thereby determine said time-lag.

Example 23. The system according to any one of examples 15 to 22, wherein the signal synchronizer is configured and operable to carry out the compensation for the clock-rate-ratio by adjusting the second clock-rate of the second signal clock in order to reduce or eliminate a difference between the second clock-rate and the first clock-rate of the first clock of the transmitter receiver utility.

Example 24. The system according to any one of examples 15 to 23, wherein the signal synchronizer is configured and operable to carry out the compensation for the clock-rate-ratio by interpolating the digitized signals according to the clock-rate-ratio, to yield the digitized signals as interpolated digital signals with samples corresponding to the first clock rate of the first clock of the transmitter receiver utility.

Example 25. The system according to any one of examples 15 to 24, wherein the signal synchronizer is configured and operable to carry out the compensation for the time lag by adjusting the phase of the second clock in order to reduce or eliminate the time lag between the first and second clock.

Example 26. The system according to any one of examples 15 to 25, wherein the signal synchronizer is configured and operable to carry out the compensation for the time lag by interpolating the digitized signals to shift their phases according to the time lag and thereby yield the digitized signals as interpolated digital signals with phases adjusted relative to the timing of the first clock of the transmitter receiver utility.

Example 27. The system according to any one of examples 15 to 26, further includes a position determination utility that is configured and operable for determining the position of the at least portion of the catheter's distal end based on the phases; and wherein the position includes at least one of a location and orientation of the at least portion of the catheter's distal end, relative to one or more reference-frame coordinates.

It should also be appreciated that the examples described above are cited by way of example, and that the present disclosure is not limited to what has been particularly shown and described hereinabove. Rather, the scope of the present disclosure includes both combinations and sub-combinations of the various features described hereinabove, as well as variations and modifications thereof, which would occur to persons of ordinary skills in the art upon reading the description of the present invention and which are not disclosed in the prior art.

1. A method to determine a position of at least a portion of a catheter's distal end, the method comprising:
by a position sensor at said at least portion of the catheter's distal end, receiving a plurality of location signals being wireless electromagnetic signals that were transmitted by a transmitter-receiver utility with respective plurality of transmission frequencies relative to a first clock-rate of a first clock associated with said transmitter-receiver utility; and
processing the location signals by a signal processor associated with a second signal clock not synchronized with said first clock and having a second clock-rate, to determine phases of the location signals relative to their respective transmissions by the transmitter-receiver utility, said processing comprises:
A. determining a clock-rate-ratio as a ratio between said first and second clock-rates;
B. determining a time-lag between said first and second signal clocks; and
C. digitizing the location signals based on said second clock to yield respective digitized signals, wherein said digitizing includes compensating for said clock-rate-ratio and compensating for said time-lag, such that frequencies and phases of said digitized signals are adjusted relative to said first signal clock of the transmitter receiver utility;
thereby determining said phases of the location signals relative to their respective transmissions by the transmitter receiver utility and enabling to determine the position of said at least a portion of a catheter's distal end based on said phases.

2. The method according to claim 1 wherein said determining of the clock-rate-ratio between said first and second clock-rates, comprises the following:
(a) providing data indicative of transmission frequency of at least one location signal of said location signals relative to the first clock-rate of the transmitter-receiver utility by which it is transmitted;
(b) processing said at least one location signal by said signal processor and determining the reception frequency thereof relative to the second clock-rate; and
thereby determining said clock-rate-ratio based on said transmission and reception frequencies.

3. The method according to claim 2 wherein said determining of the reception frequency of said at least one location signal relative to the second clock-rate comprises digitizing the location signal based on said second clock to yield a digitized signal and applying tone detection processing to the digitized signal.

4. The method according to claim 2 wherein said determining of the reception frequency of said at least one location signal relative to the second clock-rate comprises applying Clock-Derivation processing to said at least one location signal.

5. The method according to claim 4 wherein said Clock-Derivation processing comprises determining a tick-count of said second signal clock between zero-crossings of said at least one location signal and thereby determining the receipt frequency thereof relative to said second signal clock.

6. The method according to claim 1 wherein said determining of the time-lag between said first and second signal clocks, comprises the following:
(a) utilizing said clock-rate-ratio for generating, by said signal processor, a dummy signal with frequency that matches a certain transmission frequency of said transmission frequencies, and feeding said dummy signal to a receiver input of said transmitter-receiver utility, to thereby cause said transmitter-receiver utility to determine a phase difference between said dummy signal and one of said wireless electromagnetic signals that is transmitted thereby with said transmission frequency; and
(b) determining said time-lag based on said phase difference and said certain transmission frequency.

7. The method according to claim 6, wherein said feeding of said dummy signal is carried out utilizing a channel of substantially fixed latency, and wherein said fixed latency is accounted for in said determining of the time-lag.

8. The method according to claim 6, wherein said generating of the dummy signal comprises compensating for said clock-rate-ratio to thereby obtain said dummy signal with frequency matching said certain transmission frequency.

9. The method according to claim 6, comprising providing said phase difference between said dummy signal and said one of said wireless electromagnetic signals to the signal processor for said determining of the time-lag.

10. The method according to claim 1, wherein said compensating for said clock-rate-ratio comprises adjusting the second clock-rate of said second signal clock in order to reduce or eliminate a difference between said first and second clock rates.

11. The method according to claim 1, wherein said compensating for said time lag comprises adjusting the phase of said second signal clock in order to reduce or eliminate said time lag.

12. The method according to claim 1 wherein said compensating for said clock-rate-ratio comprises re-interpolating said digitized signals based on said clock-rate-ratio to yield said digitized signals with samples corresponding to a sampling rate of said first signal clock.

13. The method according to claim 1, wherein said compensating for said time lag comprises re-interpolating said digitized signals to shift their phases according to said time lag and thereby yield said digitized signals with phases adjusted relative to their respective transmissions by the transmitter receiver utility.

14. The method according to claim 1 further comprising determining said position of said at least portion of the catheter's distal end based on said phases, whereby said position comprises at least one of a location and orientation of the at least portion of the catheter's distal end relative to one or more reference-frame coordinates.

15. A system to determine a position of at least a portion of a catheter's distal end, the system comprises:
a signal processor configured and operable for connecting to a position sensor arranged at an at least a portion of a catheter's distal end, for receiving, from the position sensor, a plurality of location signals indicative of electromagnetic signals transmitted by a transmitter-receiver utility with respective plurality of transmission frequencies relative to a first clock-rate of a first clock of the transmitter-receiver utility and received by the position sensor;
wherein said signal processor comprises a second signal clock having a second clock-rate not synchronized with said first clock; and
wherein said signal processor is configured and operable to process the location signals to determine their phases relative to their respective transmissions by the transmitter-receiver utility; the signal processor comprising:
A. a clock rate processor adapted to determine a clock-rate-ratio between said first and second clock-rates;
B. a time lag processor adapted to determine a time-lag between said first and second signal clocks; and
C. a signal synchronizer configured and operably for utilizing said second clock, said clock-rate-ratio and said time-lag to digitize the location signals with compensation for said clock-rate-ratio and compensation for said time-lag, to yield respective digitized signals with frequencies and phases adjusted relative to said first signal clock of the transmitter receiver utility;
said signal processor thereby determines the phases of the location signals relative to their respective transmissions by the transmitter receiver utility and thereby enables to determine the position of said at least portion of the catheter's distal end based on said phases.

16. The system according to claim 15 wherein the clock rate processor is configured and operable to determine said clock-rate-ratio by carrying out the following:
(a) obtaining data indicative of a transmission frequency of at least one location signal of said location signals relative to the first clock-rate of the transmitter-receiver utility;
(b) processing said at least one location signal to determine a reception frequency thereof relative to the second clock-rate; and
(c) determining said clock-rate-ratio based on said transmission and reception frequencies of the at least one location signal.

17. The system according to claim 16 wherein the clock rate processor is adapted to determine the reception frequency of the at least one location signal relative to the second clock-rate by digitizing the location signal based on the clock rate of the second clock to yield a digitized signal and apply tone detection processing to the digitized signal to thereby determine the frequency of the location signal.

18. The system according to claim 16 wherein the clock rate processor comprises a Clock-Derivation processor that is adapted to determine the reception frequency of the at least one location signal relative to the second clock-rate; and wherein the Clock-Derivation processor is configured and operable to determine a tick-count of the second signal clock between zero-crossings of the at least one location signal and thereby determine the receipt frequency thereof relative to the second signal clock.

19. The system according to claim 15 wherein the time lag processor is configured and operable to determine said time-lag by carrying out the following:
(a) obtaining said clock-rate-ratio from the clock rate processor and utilizing the clock-rate-ratio to generate a dummy signal with frequency matching a certain transmission frequency of said transmission frequencies, and feed said dummy signal to a receiver input of said transmitter-receiver utility, to thereby cause the transmitter-receiver utility to determine a phase difference between said dummy signal and one of said wireless electromagnetic signals which is transmitted thereby with said transmission frequency; and
(b) obtaining said phase difference and determining said time-lag based on said phase difference and said certain transmission frequency.

20. The system according to claim 19, comprising a signal channel of substantially fixed and predetermined latency interconnected directly or indirectly between said signal processor and said transmitter receiver utility, and wherein said time lag processor is adapted to feed said dummy CW signal to said transmitter receiver utility via said channel of the substantially fixed latency, and to account for the fixed latency of said channel in determination of said time-lag.

21. The system according to claim 19, wherein said time lag processor is adapted to carry out said compensation for the clock-rate-ratio when generating said dummy signal such that said dummy signal is generated with frequency that matches said certain transmission frequency.

22. The system according to claim 19, comprising a direct or indirect connection with said transmitter receiver utility and wherein said time lag processor is adapted utilize said connection to obtain the phase difference between said dummy signal and said one of said wireless electromagnetic signals that is transmitted by the transmitter receiver utility with similar frequency as that of the dummy signal, and thereby enable determination of said time-lag.

23. The system according to claim 15, wherein the signal synchronizer is configured and operable to carry out said compensation for the clock-rate-ratio by adjusting the second clock-rate of said second signal clock in order to reduce or eliminate a difference between the second clock-rate and the first clock-rates of the first clock of the transmitter receiver utility.

24. The system according to claim 15, wherein the signal synchronizer is configured and operable to carry out said compensation for the clock-rate-ratio by interpolating the digitized signals according to said clock-rate-ratio, to yield said digitized signals as interpolated digital signals with samples corresponding to the first clock rate of the first clock of the transmitter receiver utility.

25. The system according to claim 15, wherein the signal synchronizer is configured and operable to carry out said compensation for the time lag by adjusting the phase of said second clock in order to reduce or eliminate said time lag between the first and second clock.

26. The system according to claim 15, wherein the signal synchronizer is configured and operable to carry out said compensation for the time lag by interpolating the digitized signals to shift their phases according to said time lag and thereby yield said digitized signals as interpolated digital signals with phases adjusted relative to the timing of said first clock of the transmitter receiver utility.

27. The system according to claim 15, further comprising a position determination utility that is configured and operable for determining the position of said at least portion of the catheter's distal end based on said phases; and wherein said position comprises at least one of a location and orientation of the at least portion of the catheter's distal end, relative to one or more reference-frame coordinates.
